(19) United States
(12) Patent Application Publication
(10) Pub. No.: US 2013/0180037 A1
(43) Pub. Date: Jul. 18, 2013

(54) SIMPLE AND HANDY SNOW GOGGLES

(71) Applicant: CHIH-HUNG WANG, TAINAN CITY (TW)

(72) Inventor: CHIH-HUNG WANG, TAINAN CITY (TW)

(21) Appl. No.: 13/348,720

(22) Filed: Jan. 12, 2012

(51) Int. Cl. A61F 9/02

(52) U.S. Cl. 2/431

(57) ABSTRACT

This invention relates to a simple and handy snow goggles in which an elastic face-fitting part is attached to the rear side face of a lens along the periphery. Both sides of the lens are extended outward to form extension portions on which slots are provided for assembly with gaskets. A strap is linked between the two gaskets. In this manner, a simple and handy snow goggles with frameless structure and easiness for lens replacement can be obtained.

### BACKGROUND OF INVENTION

1. Field of the Invention

The present invention relates to a simple and handy snow goggles, more particularly to a simple and handy snow goggles having frameless structure and easiness for lens replacement.

2. Brief Description of Prior Art

Skiing is a thrilling and exciting activity. As temperature in skiing field is quite low and skiing has a certain speed, basic equipment for a skier includes gloves, hat and snow goggles, in addition to warmer clothing.

Currently, snow goggles available on market all have a frame, i.e., a lens is inlaid in the frame and a face-fitting component is provided on the side adjacent to user's face. However to some people, skiing activity is accidental rather than frequent exercise, but even if so, to wear a snow goggles is a must in skiing. Therefore, consumers cannot but to purchase snow goggles even for a single attendance of skiing activity. As the snow goggles available on market all have a frame as aforementioned, a separate mold has to be fabricated for producing the frame. This will result in cost up and hence higher retail price too, and thus, out with the old and in with the new may not be feasible for customers, as spending more money to purchase an expensive snow goggles for the possible sole attendance of skiing activity is ultimately not cost-effective. In terms of manufactures, to change the shape of lens means to change the shape of the frame simultaneously and hence mold for producing the frame has to be made separately. Accordingly, to change the shape of snow goggles requires much time and work which causes higher expense. At the same time, the shape of lens is out of full play due to the frame mold fabrication problem.

### SUMMARY OF INVENTION

The primary object of the present invention is to provide a simple and handy snow goggles with frameless structure and easiness for lens replacement which can be done at any time depending on usage, so that cleanness and hygiene on the usage of the lens can be ensured. Simultaneously, the shape of lens is no longer given in to the shape of the frame so that the design of lens can be fully diversified.

The above object of the present invention can be achieved by the following specific means.

A simple and handy snow goggles in which an elastic face-fitting part is attached to the rear side face of a lens along the periphery. Both sides of the lens are extended outward to form extension portions on which slots are provided for assembly with gaskets. A strap is linked between the two gaskets.

Configuring like this, the elastic face-fitting part attached to the rear side face of a lens along the periphery can be fitted closely on user's face, and then the strap encircles and stretches around user's head so as to wear the snow goggles fixedly on user's face.

According to the snow goggles of the present invention, the two gaskets assembled within the two slots of the lens respectively have breaches so that the gaskets almost have a C-shape. The ends having breaches possess elasticity for compression so that the gaskets can be inlaid into the slots easily.

According to the snow goggles of the present invention, insertion grooves are further provided along the peripheries of the two gaskets. The peripheral edges of the slots on the lens can be inserted into the insertion grooves so as to facilitate the attachment of the gaskets on the lens.

According to the snow goggles of the present invention, auxiliary sheets are provided separately at two ends on the outside surface of the lens. Each auxiliary sheet has a slot corresponding to the slot of the lens so that the gaskets can be inlaid within both the slots to clamp the auxiliary sheet in place at the same time.

According to the snow goggles of the present invention, auxiliary sheets further have patterns provided on the surface thereof. The patterns can be drawings or characters formed by carving, printing or adhering.

### BRIEF DESCRIPTION OF THE DRAWINGS

FIG. 1 is a perspective exploded enlarged view showing the lens and the gaskets of the simple and handy snow goggles of the present invention.

FIG. 2 is a perspective assembled schematic view showing another simple and handy snow goggles of the present invention.

FIG. 3 is a perspective assembled view showing another simple and handy snow goggles of the present invention.

FIG. 4 is a partial enlarged schematic view of another simple and handy snow goggles of the present invention showing that the gaskets is assembling on the lens.

FIG. 5 is a partial enlarged schematic view of another simple and handy snow goggles of the present invention showing that the gaskets is assembled on the lens.

FIG. 6 is a schematic view of another simple and handy snow goggles of the present invention showing that the gaskets is assembled on the lens.

FIG. 7 is a schematic view of still another simple and handy snow goggles of the present invention showing that the auxiliary sheets is combined with the lens.

### DETAILED DESCRIPTION OF PREFERRED EMBODIMENTS

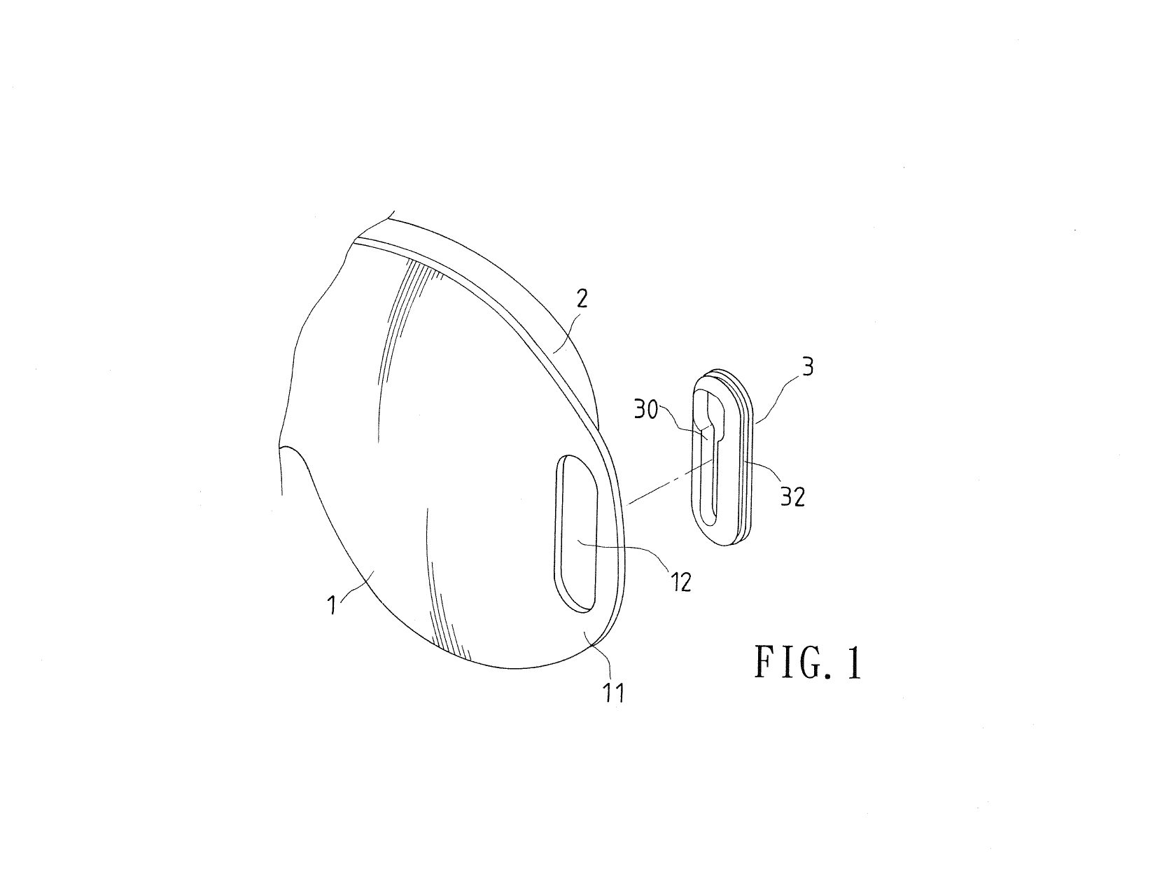

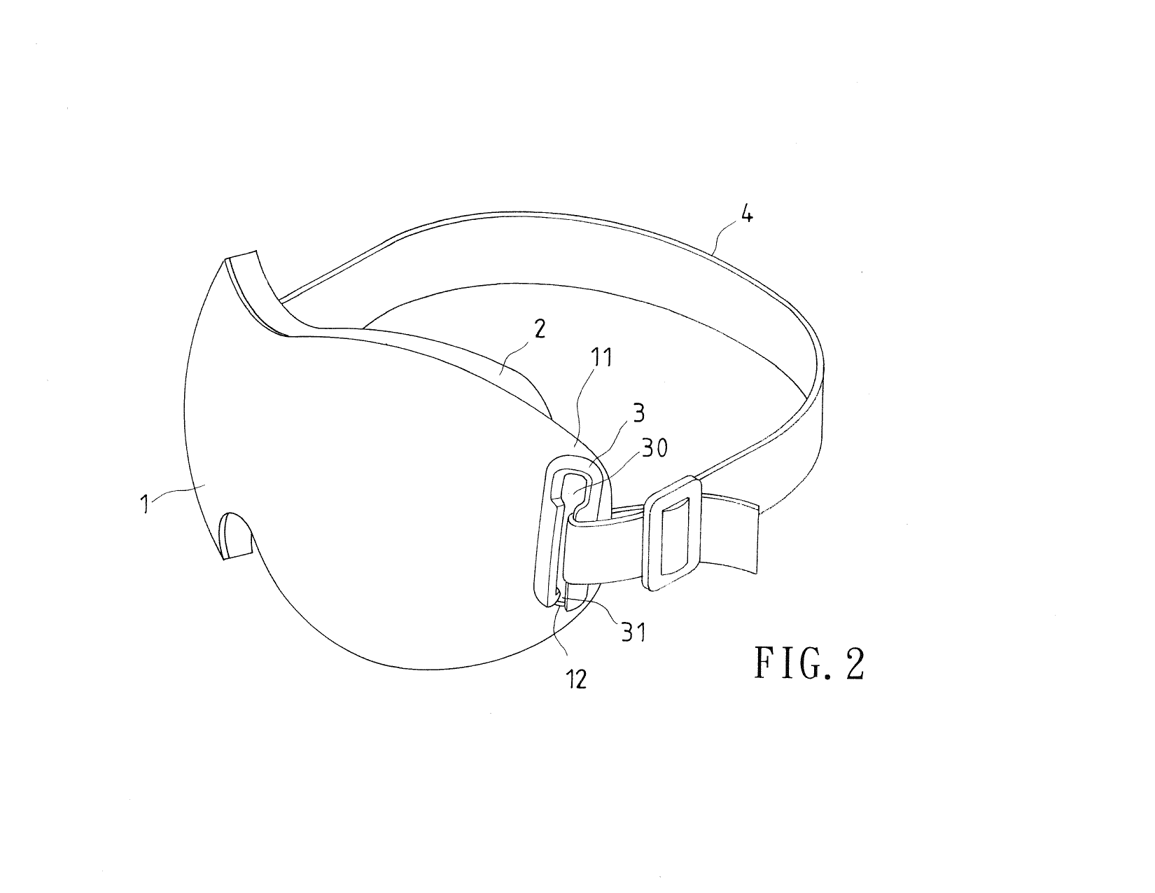

Referring to FIGS. 1 and 2, the simple and handy snow goggles of the present invention comprises a lens (1), an elastic face-fitting part (2), two gaskets (3) and a strap (4).

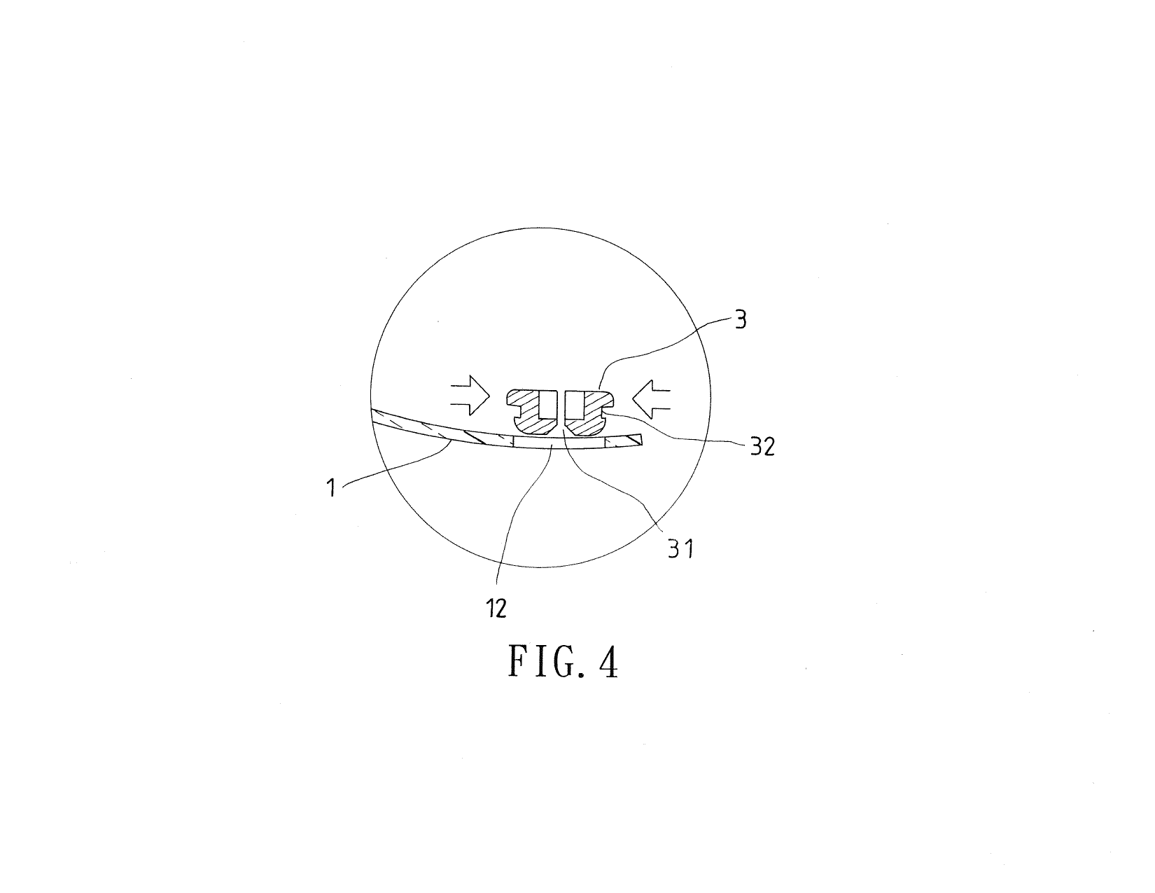

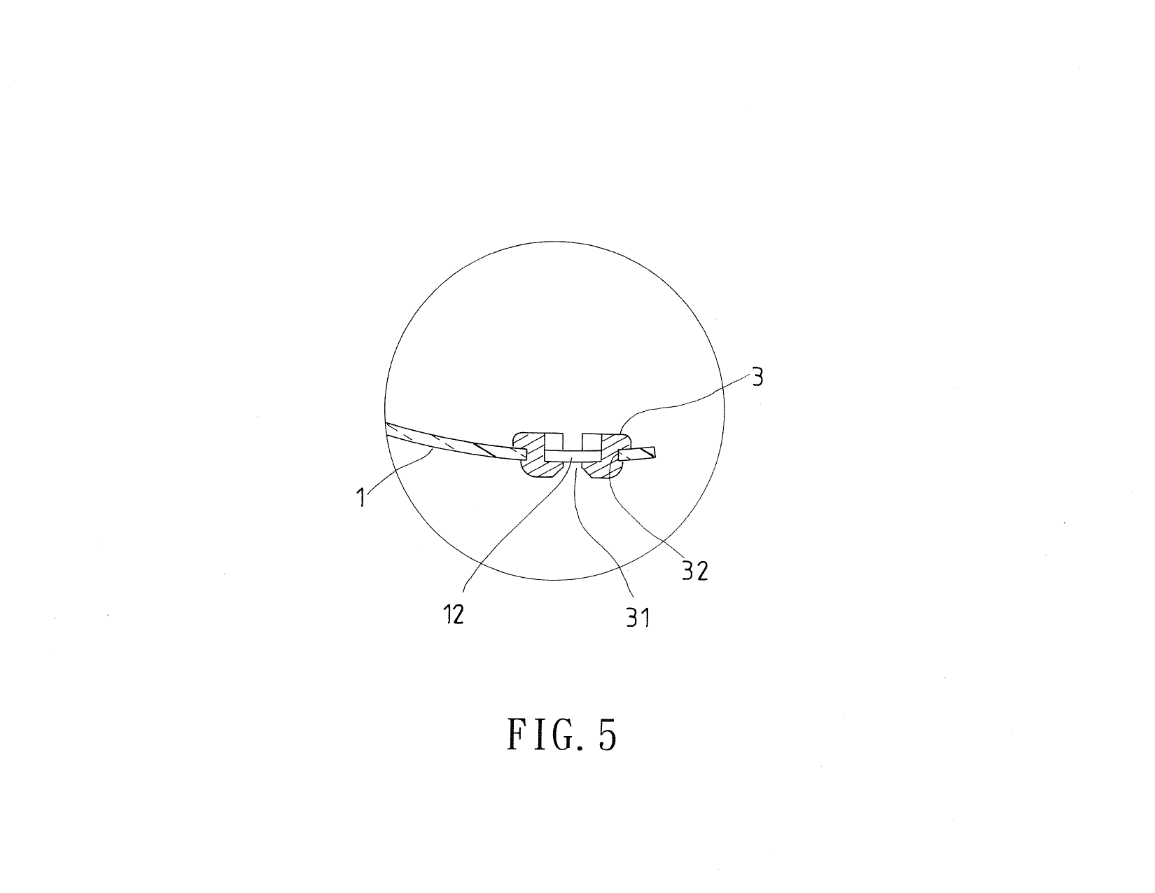

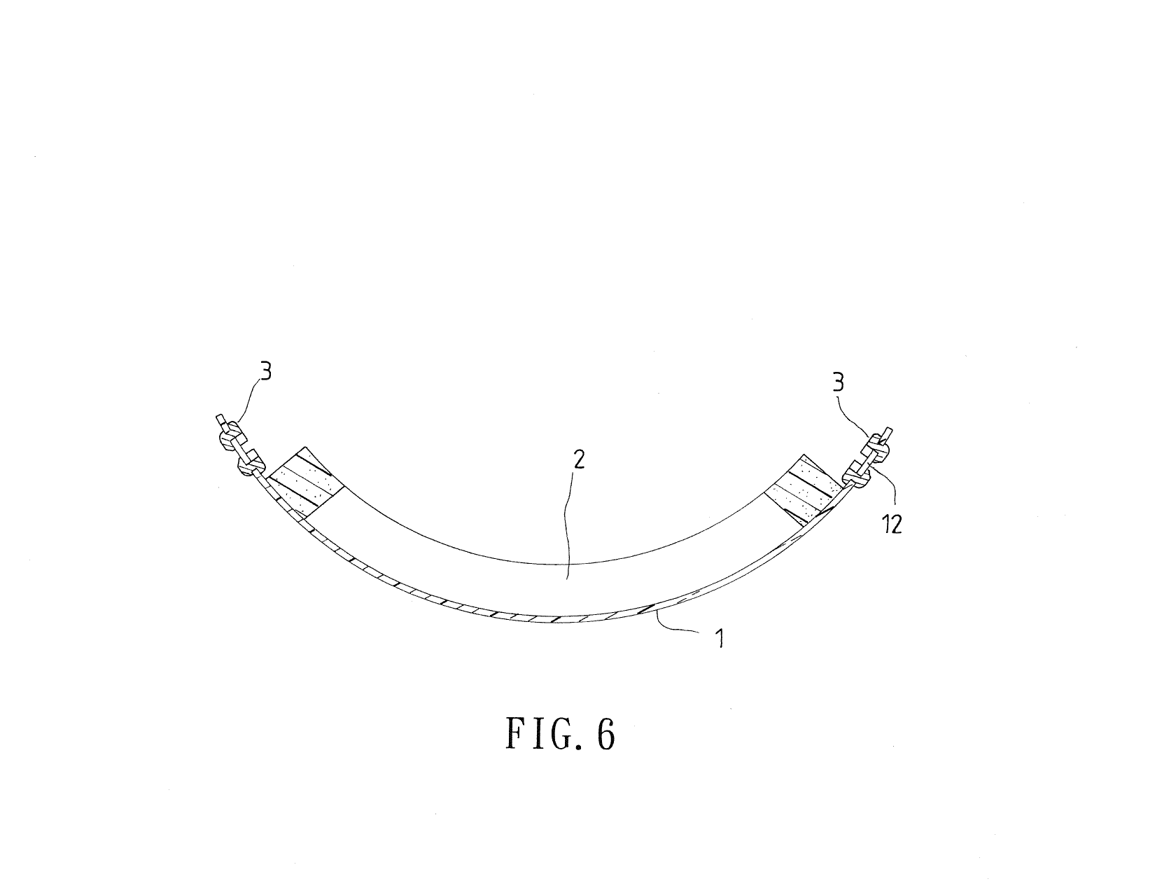

The lens (1) is made to be an integral type lens structure in which the elastic face-fitting part (2) is attached to the rear side face of the lens (1) along the periphery ant both sides of the lens (1) is extended outward to form extension portions (11). Each extension portion (11) is provided with a slot (12) which should not be covered by the elastic face-fitting part (2) after assembly of the same, as shown in FIG. 6. The two gaskets (3) are respectively inlaid within the two slots (12), as shown in FIGS. 4 and 5, and each of the gaskets (3) has a through hole (30) for the passing through of the strap (4). FIG. 2 reveals that the strap (4) is linked between the two gaskets (3) and the strap (4) has elasticity.

When wearing the snow goggles, the lens (1) is disposed to confront with user's eyes on user's face and the elastic face-fitting part (2) attached to the rear side face of the lens (1) along the periphery is fitted closely on user's face. Then, the strap (4) encircles and stretches around user's head so as to wear the snow goggles fixedly on user's face.

According to the snow goggles of the present invention, there is no frame provided to encircle around the lens (1), but the lens (1) can still fit tightly on user's face through the elastic face-fitting part (2) attached to the rear side face of the lens (1) along the periphery. Moreover, the snow goggles with frameless design can lower the cost and hence is advantageous to render expedient usage for those who have no snow goggles on hand in large-scale public occasion (for example, a ski field). Furthermore, the shape of the lens (1) is no longer given in to the shape of the frame so that the design of lens can be fully diversified.

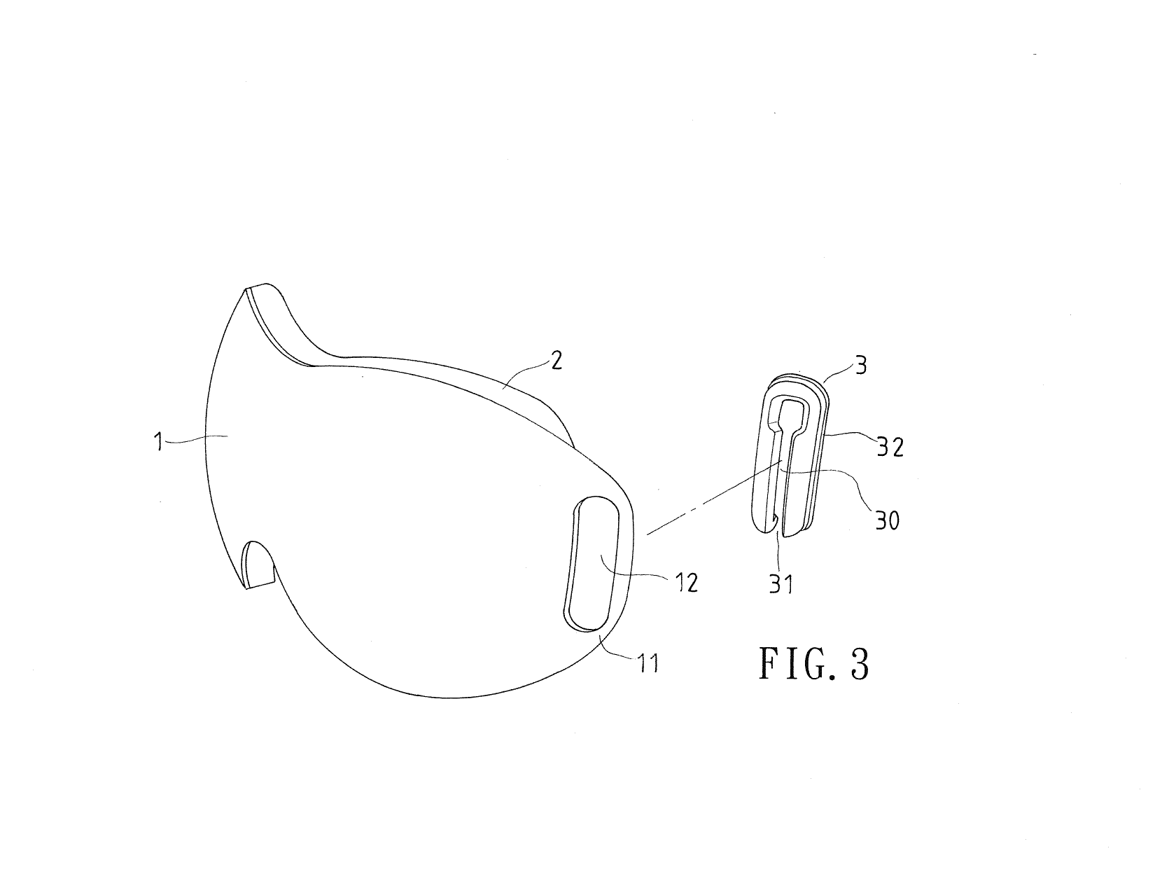

Moreover, the two gaskets (3) assembled within the two slots (12) of the lens (1) respectively have breaches (31) provided at one ends so that the gaskets (3) almost have a C-shape, as shown in FIGS. 3 and 5. The ends having breaches (31) possess elasticity for compression so that the gaskets (3) can be inlaid into the slots easily by compressing the gaskets (3). Further, insertion grooves (32) are provided along the peripheries of the two gaskets (3), and the peripheral edges of the slots (12) of the lens (1) can be inserted into the insertion grooves (32) so as to facilitate the attachment of the gaskets (3) on the lens (1) and to facilitate the detachment of the gaskets (3) from the slots (12) for replacement of a new lens (1).

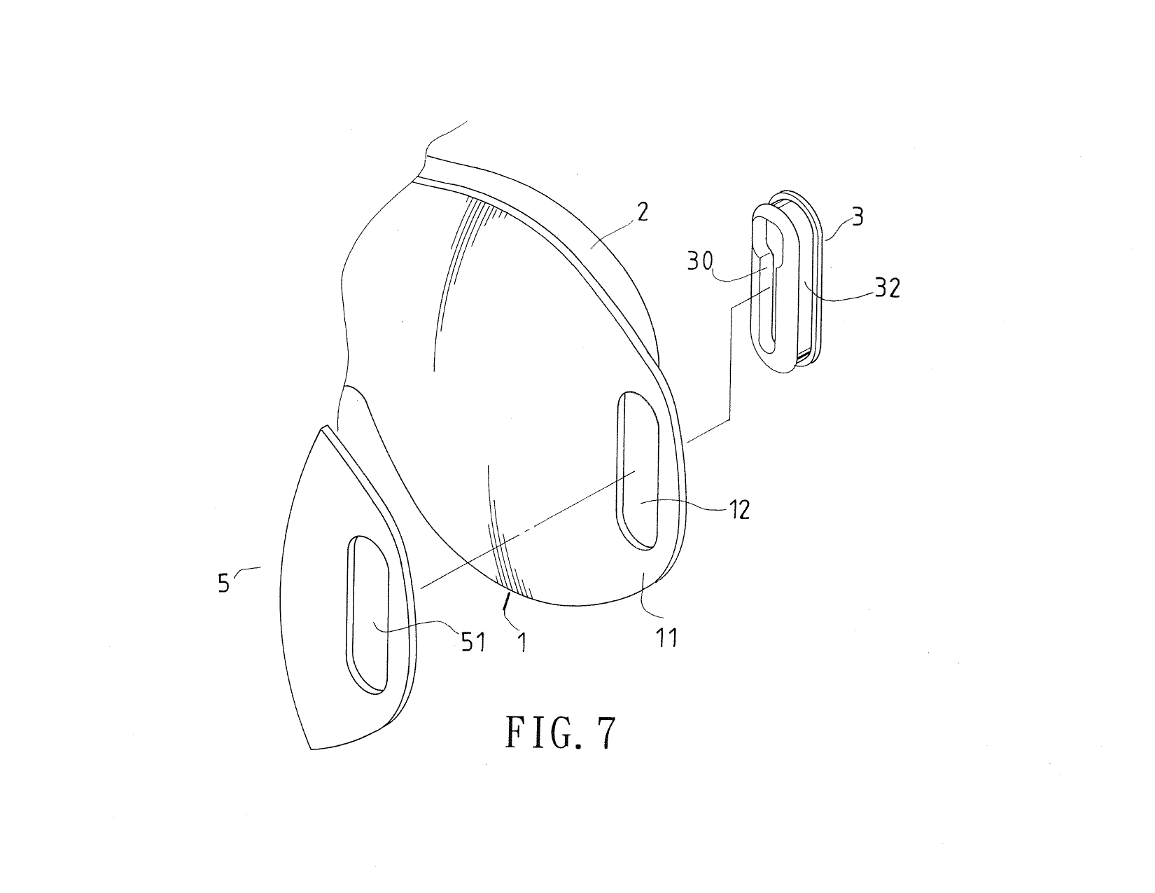

Referring to FIG. 7, the snow goggles of the present invention has auxiliary sheets (5) provided separately at two ends on the outside surface of the lens (1). Each auxiliary sheet (5) has a hole (51) corresponding to the slot (12) of the lens (1) so that the gaskets (3) can be inlaid within both the slots (12) and the hole (51) to clamp the auxiliary sheet (5) in place at the same time.

Moreover, the auxiliary sheets (5) may have patterns (not shown in figures) provided on the surface thereof The patterns can be drawings or characters formed by anyone method of carving, printing or adhering or their combination.

Based on foregoing, the snow goggles of the present invention at least has advantages list below.

1. As the snow goggles of the present invention is frameless, the cost the snow goggles can be lowered and hence the snow goggles can provide expedient usage for users.
2. In order to replace the lens, it only needs to release the loop of the strap tightened on the gaskets and then remove the gaskets from the slots. Therefore, the replacement of the lens is very easy.
3. As the lens of the snow goggles can be readily replaced depending on usage and replacement is easy, the lens can be kept in clean and hygiene status all the time.
4. As the snow goggles of the present invention is frameless, the shape of the lens is no longer given in to the shape of the frame so that the design of lens can be fully diversified.
5. With the addition of the auxiliary sheets, the snow goggles of the present invention can increase its variations and thus its appearance appeal.

What is claimed is:

1. A simple and handy snow goggles, wherein an elastic face-fitting part is attached to the rear side face of a lens along the periphery;
both sides of the lens being extended outward to form extension portions, two slots being provided respectively on said extension portions for assembly with gaskets; a strap being linked between said two gaskets.

2. The simple and handy snow goggles as claimed in claim 1, wherein said lens is an integral type lens structure.

3. The simple and handy snow goggles as claimed in claim 1, wherein each of said gaskets has a breach so that said gasket is formed almost as a C shape.

4. The simple and handy snow goggles as claimed in claim 3, wherein insertion grooves are provided along the outer periphery of said gaskets for the insertion of the peripheral edges of slots provided on said lens.

5. The simple and handy snow goggles as claimed in claim 1, wherein auxiliary sheets are provided separately at two ends on the outer surface of said lens, each auxiliary sheet having a slot corresponding to said slot of the lens so that said gaskets can be inlaid within both said slots to clamp said auxiliary sheet in place.

6. The simple and handy snow goggles as claimed in claim 5, wherein said auxiliary sheets have patterns provided on the surfaces thereof.

7. The simple and handy snow goggles as claimed in claim 6, wherein said patterns can be drawings or characters or their combination formed by anyone of or combination of carving, printing or adhering.

8. The simple and handy snow goggles as claimed in claim 5, wherein said lens is an integral type lens structure.

9. The simple and handy snow goggles as claimed in claim 5, wherein each of said gaskets has a breach so that said gasket is formed almost as a C shape.

10. The simple and handy snow goggles as claimed in claim 9, wherein insertion grooves are provided along the outer periphery of said gaskets for the insertion of the peripheral edges of slots provided on said lens.
